(19) United States
(12) Patent Application Publication
(10) Pub. No.: US 2021/0049434 A1
(43) Pub. Date: Feb. 18, 2021

(54) SYSTEMS AND METHODS FOR MONITORING MOTION AND APPLICATIONS THEREOF

(71) Applicant: INTERNATIONAL THERMODYNE, INC., Charlotte, NC (US)

(72) Inventors: Katherine Holman HUGHES, Huntersville, NC (US); John Paul SOLITARIO, Charlotte, NC (US); Timothy Alan RISSER, Charlotte, NC (US)

(21) Appl. No.: 17/049,832

(22) PCT Filed: Apr. 25, 2019
(86) PCT No.: PCT/US19/29149

§ 371 Date: Oct. 22, 2020

Related U.S. Application Data
(60) Provisional application No. 62/662,575, filed on Apr. 25, 2018.

(51) Int. Cl. G06K 19/07; A61B 5/11; A61B 5/00; G16H 40/67; G06Q 50/28

(52) U.S. Cl. CPC G06K 19/0705; A61B 5/11; A61B 5/0002; A61B 5/6804; G06K 19/0711; H02J 7/0047; G16H 40/67; G06Q 50/28; A61B 2560/0214; A61B 2503/40; G06K 19/0727

(57) ABSTRACT

In one aspect, systems for monitoring motion are described herein. A system for monitoring motion comprises a battery component in electrical communication with an energy storage component via a momentary operation switch that is closed by movement of the system, The system also comprises a signaling component providing a signal in response to the energy storage component reaching a minimum energy threshold.

### RELATED APPLICATION DATA

The present application claims priority pursuant to 35 U.S.C. § 119(e) to United States Provisional Patent Application Ser. No. 62/662,575 filed Apr. 25, 2018 which is incorporated herein by reference in its entirety.

### FIELD

The present invention relates to systems and methods for monitoring motion of various subjects in various environments.

### BACKGROUND

Monitoring workers and inventory in the commercial setting is an important aspect of a successful business. Tracking inventory movement and location, for example, can facilitate supply chain management and provide enhanced efficiencies of product sale and delivery. Additionally, monitoring employee movement in warehouses or construction sites can assist in avoiding accidents and workplace injuries. However, current monitoring systems often require advanced electronics and associated power sources. Once the systems are initiated, the power sources run in a continuous mode. In the absence of careful diligence, the power sources can exhaust, thereby terminating monitoring capabilities of the system. This is a significant problem for management of inventory having an extended shelf-life or inventory and equipment that has been placed in long-term storage.

### SUMMARY

In view of these disadvantages, systems and methods for monitoring motion are described herein, which can mitigate power consumption. Briefly, a system for monitoring motion comprises a battery component in electrical communication with an energy storage component via a momentary operation switch that is closed by movement of the system. The system also comprises a signaling component providing a signal in response to the energy storage component reaching a minimum energy threshold.

In another aspect, a method of monitoring motion comprises coupling a monitoring system to a subject, wherein the monitoring system comprises a signaling component and a battery component, wherein the battery component is in electrical communication with an energy storage component via a momentary operation switch. The momentary operation switch is closed in response to motion or movement by the subject. Closing of the momentary switch permits charging of the energy storage component by the battery component. In the absence of motion or movement by the subject, the momentary switch returns to the normal open position and charging of the energy storage component ceases. A signal is emitted by the signaling component in response to the energy storage component reaching a minimum energy threshold.

These and other embodiments are described further in the following detailed description.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

In one aspect, a system for monitoring motion comprises a battery component in electrical communication with an energy storage component via a momentary operation switch that is closed by movement or motion of the system. When closed, the battery component may charge the energy storage component. When movement or motion of the system ceases, the momentary operation switch returns to the normal open positon and energy transfer between the battery component and storage component is terminated. This energy transfer or charging cycle between the battery component and storage component can be repeated any number of times in response to movement or motion of the system. Each charging cycle brings the storage component closer to meeting the minimum energy threshold required for signal generation by the signaling component. As described further herein, the system can be coupled to a subject that is responsible for inducing motion of the system. The subject, for example, can be a human, animal, equipment or inventory. Moreover, the momentary operation switch can have any design not inconsistent with the objectives of the present invention. In some embodiments, for example, the momentary operation switch is a spring switch.

The system also comprises a signaling component providing a signal in response to the energy storage component reaching a minimum energy threshold. In some embodiments, the minimum energy threshold is based, at least in part, on voltage of the battery component. The minimum energy threshold can be a voltage ratio of the energy storage component and battery component, as set forth in equation (1):

$${E}_{m} = \frac{{V}_{esc}}{Vbc} \qquad ( 1 )$$

wherein Eₘ is the minimum energy threshold, V_{esc} is the energy storage component voltage and V_{bc} is the battery component voltage. The voltage ratio of the energy storage component and battery component can be set to any desired value. In some embodiments, the voltage ratio is greater than zero, but less than or equal to 1. Alternatively, the voltage ratio can be greater than 1. Systems described herein can employ a comparator component to determine the voltage ratio between the energy storage component and battery component. In other embodiments, the minimum energy threshold can correspond to an amount of energy in the storage component. The energy storage component, for example, may comprise one or more capacitors, wherein the minimum energy threshold is a function of capacitance. A capacitance value can be set as the energy threshold. In other embodiments, the energy storage component can comprise one or more batteries, wherein the minimum energy threshold is a function of battery voltage.

The minimum energy threshold can be set at any desired value. Considerations governing the energy threshold can include, but are not limited to, the type and amount of motion or movement experienced by the system as well as the frequency of the motion. If the motion experienced by the system is large and frequent, the minimum energy threshold can be raised to avoid frequent signal generation. If the amount of motion is small and/or infrequent, the energy threshold minimum can be lowered to enhance sensitivity. The energy minimum threshold can be largely dependent on the identity of the subject to which the monitoring system is coupled.

When the minimum energy threshold is reached by the energy storage component, the signaling component is powered and a signal is emitted. The signaling component, in some embodiments, is powered by the energy storage component. Upon reaching the minimum energy threshold, one or more capacitors of the storage component can discharge and power the signaling component. In other embodiments, the signaling component can be powered by the battery component alone or in combination with the storage component. As discussed further herein, the energy minimum threshold may be set at low levels to enhance sensitivity to motion or movement of the system. Such low levels may be insufficient to power the signaling component. Under these circumstances, the battery component may join with the energy storage component to power the signaling component. Alternatively, the battery component alone may power the signaling component or the signaling component can be powered by an additional power source of the system.

Signals provided by the signaling component can be in the form of radio waves or other electromagnetic radiation. In some embodiments, the radio waves can be in the Ultra High Frequency (UHF) band, generally having a frequency of 300 MHz to 3 GHz. For example, BLUETOOTH® or BLUETOOTH® low energy platforms and associated apparatus can be used as signaling components, in some embodiments. Signal generated by the signaling component can be transmitted to one or more electronic devices. Signal can be transmitted to any electronic device not inconsistent with the objectives of the present invention. Suitable electronic devices include, but are not limited to, cellular phones, smart phones, tablets, servers and/or computers. The signal can contain any desired information. In some embodiments, the signal is a simple beacon indicating that the system has experienced sufficient motion to meet the minimum energy threshold. In other embodiments, the signal may comprise information related to location and/or other physical condition of the system. In further embodiments, the signaling component can provide an audible signal and/or visual signal. A speaker and/or light emitting diode, for example, can signal when certain event criteria have been met.

Systems described herein can comprise one or more regions for coupling to a subject for monitoring motion of the subject. In some embodiments, a system can comprise fasteners, clips or other structures for engaging the subject. A system, for example, can comprise adhesive surface(s) for interfacing with a subject. In further embodiments, the system may be placed in a housing, and the housing is coupled the subject. The system can be part of an identification tag or labeling for the subject. Subjects can include products, assets, inventory and/or equipment. Equipment includes, but is not limited to, personal protection equipment, office equipment, scientific equipment, construction equipment and communications equipment. In some embodiments, subjects to which the monitoring systems are coupled include people or animals

Systems described herein offer the unique ability to continuously monitor various subjects without a continuous expenditure of energy. Prior monitoring systems require continuous battery use. In contrast, systems described herein only consume energy during movement or motion of the subject. In states of rest, the motion monitoring systems can remain in a non-powered state, since the momentary operation switch is in the normal open position.

When motion of the system occurs, the momentary operation switch can close permitting charging of the storage component by the battery component. When the energy storage component meets the required energy threshold, a signal is generated by the signaling component. This architecture can greatly extend battery life, thereby permitting monitoring of subjects over substantial periods of time without power loss. This is particularly advantageous for inventory or products with long shelf lives and/or or products that have been warehoused or archived.

In another aspect, a method of monitoring motion comprises coupling a monitoring system to a subject, wherein the monitoring system comprises a signaling component and a battery component, wherein the battery component is in electrical communication with an energy storage component via a momentary operation switch. The momentary operation switch is closed in response to motion or movement by the subject. Closing of the momentary switch permits charging of the energy storage component by the battery component. In the absence of motion or movement by the subject, the momentary switch returns to the open position and charging of the energy storage component ceases. A signal is emitted by the signaling component in response to the energy storage component reaching a minimum energy threshold. Systems of methods described herein can comprise any of the properties, features and/or architectures described hereinabove.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without-departing from the spirit and scope of the invention.

1. A system for monitoring motion comprising:
a battery component in electrical communication with an energy storage component via a momentary operation switch that is closed by movement of the system; and
a signaling component providing a signal in response to the energy storage component reaching a minimum energy threshold.

2. The system of claim 1, wherein the minimum energy threshold is based on voltage of the battery component.

3. The system of claim 2, wherein the minimum energy threshold is a ratio of energy storage component voltage to battery voltage.

4. The system of claim 3, wherein the ratio is less than or equal to 1.

5. The system of claim 3, wherein the ratio is greater than 1.

6. The system of claim 1, wherein the signaling component is powered by the energy storage component.

7. The system of claim 1, wherein the energy storage component comprises one or more capacitors.

8. The system of claim 3 further comprising a comparator component for determining the voltage ratio of the energy storage component and battery component.

9. The system of claim 1, wherein the signaling component employs electromagnetic radiation for communication with one or more electronic devices external to the monitoring system.

10. The system of claim 9, wherein the electromagnetic radiation comprises radio waves.

11. The system of claim 10, wherein the radio waves have a frequency of 300 MHz to 3 GHz.

12. The system of claim 9, wherein the one or more electronic devices comprise a cellular phone, computer, tablet, server, global positioning system or combinations thereof.

13. The system of claim 1 further comprising a region for coupling to apparel, machinery, goods or equipment.

14. A method of monitoring motion comprising:
coupling a monitoring system to a subject, the monitoring system comprising a signaling component and a battery component, the battery component in electrical communication with an energy storage component via a momentary operation switch;
closing the momentary operation switch in response to motion or movement by the subject to charge the energy storage component with the battery component; and
emitting a signal with the signaling component in response to the energy storage component reaching a minimum energy threshold.

15. The method of claim 14, wherein the minimum energy threshold is based on voltage of the battery component.

16. The method of claim 15, wherein the minimum energy threshold is a ratio of energy storage component voltage to battery voltage.

17. The method of claim 16, wherein the ratio is less than or equal to 1.

18. The method of claim 16, wherein the ratio is greater than 1.

19. The method of claim 14, wherein the energy storage component comprises one or more capacitors, batteries or combinations thereof.

20. The method of claim 16, wherein the system further comprises a comparator component for determining the voltage ratio of the energy storage component and battery component.

21. The method of claim 14, wherein the energy storage component powers the signaling component.

22. The method of claim 14, wherein the signal comprises electromagnetic radiation.

23. The method of claim 22, wherein the electromagnetic radiation comprises radio waves.

24. The method of claim 14 further comprising receiving the signal with one or more electronic devices.

25. The method of claim 25, wherein the one or more electronic devices comprise a cellular phone, computer, tablet, server, global positioning system or combinations thereof.

26. The method of claim 14, wherein the subject is selected from the group consisting of a person, animal, equipment, products and inventory.
